(19) United States
(12) Patent Application Publication
(10) Pub. No.: US 2026/0090855 A1
(43) Pub. Date: Apr. 2, 2026

(54) COORDINATED MOVEMENTS OF ROBOTIC TOOLS

(71) Applicant: Auris Health, Inc., Santa Clara, CA (US)

(72) Inventor: Alexander Tarek Hassan, San Francisco, CA (US)

(21) Appl. No.: 19/410,925

(22) Filed: Dec. 5, 2025

Related U.S. Application Data
(63) Continuation of application No. 17/022,563, filed on Sep. 16, 2020.
(60) Provisional application No. 62/902,872, filed on Sep. 19, 2019.

(51) Int. Cl. A61B 34/37; A61B 17/00; A61B 34/20; A61B 34/30; A61B 50/13; B25J 9/16; B25J 13/06

(52) U.S. Cl. CPC A61B 34/37; B25J 9/1682; B25J 9/1689; B25J 9/1697; B25J 13/065; A61B 2017/00199; A61B 2017/00973; A61B 2034/2051; A61B 2034/301; A61B 2034/302; A61B 50/13

(57) ABSTRACT

Provided is a robotic medical system and related methods for performing coordinated movements of robotic tools inserted within a patient through multiple separate ports and maintaining remote centers of motion during the coordinated movements. In a first control mode, a first robotic tool is moved (e.g., a camera) using a first controller and multiple robotic tools are automatically moved in coordinated movements with the first robotic tool. In a second control mode, a robotic camera is controlled using a first controller and multiple robotic tools are automatically moved in coordinated movements to stay within a field of view of the robotic camera. In a third control mode, multiple robotic tools are moved in automatic coordinated movements using a first controller and a robotic camera is controlled using a second controller.

### CROSS REFERENCE TO RELATED APPLICATIONS

This application is a continuation of U.S. Non-Provisional application Ser. No. 17/022,563, filed Sep. 16, 2020, which claims the benefit of priority to U.S. Provisional Application No. 62/902,872, filed Sep. 19, 2019, the entire contents of each of which are incorporated by reference herein.

### TECHNICAL FIELD

The systems and methods disclosed herein are directed to coordinated movement of robotic tools, and more particularly to coordinated movement between one or more robotically controlled instruments and/or cameras.

### BACKGROUND

Various medical procedures may be performed using a robotic medical system to control the insertion and/or manipulation of one or more medical instruments. For certain medical conditions, one or more internal worksites must be reached to fully treat the medical condition. The robotic medical system may include one or more robotic arms or any other instrument positioning device(s). The robotic medical system may also include a controller used to control the positioning of the instrument(s) during each of the procedures via the manipulation of the robotic arm(s) and/or instrument positioning device(s).

### SUMMARY

Some surgeries require procedures at more than one workspace within the patient's body. For example, during a total colectomy, a physician will need to perform surgical tasks in all four quadrants of a patient's abdomen. In existing systems, the control console allows control of either two instruments or a camera. As a result, moving all three tools (e.g., two instruments and a camera) from worksite to worksite can be onerous, time consuming and hazardous for the patient. The disclosure includes systems and methods that allow automatic coordinated motion between robotic tools (e.g., instruments and/or cameras). Accordingly, coordinated movement of robotic tools between worksites can be performed more conveniently and with less hazard to the patient.

According to one aspect, a robotic surgical system includes a plurality of robotic arms and a control unit configured to control movement of each of the robotic arms. A first tool is associated with a first robotic arm of the plurality of robotic arms. A second tool is associated with a second robotic arm of the plurality of robotic arms. A movement of the first robotic arm and the first tool by the control unit results in a coordinated movement of the second robotic arm and the second tool. The first robotic arm maintains a first remote center of motion during the movement and the second robotic arm maintains a second remote center of motion during the coordinated movement.

In another aspect, a robotic surgery method includes moving a first tool associated with a first robotic arm using a control unit and moving a second tool associated with a second robotic arm. The movement of the second robotic arm and the second tool is coordinated with the movement of the first robotic arm and the first tool. A first remote center of motion is maintained during the movement of the first robotic arm and the first tool. A second remote center of motion is maintained during the coordinated movement of the second robotic arm and the second tool.

In another aspect, a robotic surgical system includes a plurality of robotic arms and a control unit configured to control movement of each of the robotic arms. A first tool is associated with a first robotic arm of the plurality of robotic arms. A second tool is associated with a second robotic arm of the plurality of robotic arms. A movement of the first robotic arm and the first tool by the control unit results in a coordinated movement of the second robotic arm and the second tool.

In another aspect, a robotic surgery method includes inserting a rigid tool associated with a first robotic arm into a patient through a first port and inserting a flexible tool associated with a second robotic arm through a natural orifice of the patient. The rigid tool and the first robotic arm are moved using a control unit. The flexible tool moves in a coordinated movement with the movement of the rigid tool. A remote center of motion of the rigid tool is maintained during the coordinated movement.

In another aspect, a robotic surgical system includes a plurality of robotic arms and a control unit configured to control movement of each of the robotic arms. A first tool is associated with a first robotic arm of the plurality of robotic arms and has a first remote center of motion. A second tool is associated with a second robotic arm of the plurality of robotic arms and has a second remote center of motion. The robotic surgical system includes a processor and at least one computer-readable memory in communication with the processor. The memory has stored thereon computer-executable instructions to cause the processor to: receive an input at the control unit, cause a first movement of the first robotic arm and the first tool based on the input, coordinate a second movement of the second robotic arm and the second tool with the first movement, cause the first tool to maintain the first remote center of motion during the first movement of the first robotic arm, and cause the second tool maintain the second remote center of motion during the coordinated second movement of the second robotic arm.

In another aspect or a method operable by a robotic surgical system, the method includes moving a first tool is associated with a first robotic arm of a plurality of robotic arms based on an input received at a control unit, moving a second tool is associated with a second robotic arm of the plurality of robotic arms based on the input, coordinating movement of the second robotic arm and the second tool with the movement of the first robotic arm and the first tool, and maintaining remote centers of motion for the first and second tools during the movement and the coordinated movement.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy.

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopic procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopic procedure.

FIG. 9 illustrates an embodiment of a table-based robotic system configured for a laparoscopic procedure.

FIG. 10 illustrates an embodiment of the table-based robotic system of FIG. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 illustrates an embodiment of a robotic medical system.

FIG. 22 illustrates a pair of robotic arms and robotic tools movable about remote centers of motion.

FIG. 23 illustrates a schematic diagram of a robotic tool.

FIG. 24A illustrates five robotic tools inserted within a patient's abdomen as part of a medical procedure.

FIG. 24B illustrates the five robotic tools positioned at a first worksite within the patient's abdomen.

FIG. 24C illustrates the five robotic tools positioned at a second worksite within the patient's abdomen.

FIG. 25A-B illustrate four robotic instruments and a robotic camera.

FIG. 26 illustrates an example of a console including one or more interfaces for controlling robotic arms in accordance with aspects of this disclosure.

FIG. 27 is a flowchart illustrating an example method operable by a robotic system for performing medical procedures including coordinate motion between robotic tools.

FIG. 28 illustrates an example of a bed-based robotic system for performing concomitant procedures in accordance with aspects of this disclosure.

FIG. 29A-B illustrate coordinated movement of one or more rigid robotic tools and a flexible robotic tool.

FIG. 30 is a flowchart illustrating an example method operable by a robotic system for performing concomitant medical procedures including coordinated movement between one or more rigid robotic tools and a flexible robotic tool.

### DETAILED DESCRIPTION

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

In addition to performing the breadth of procedures, the system may provide additional benefits, such as enhanced imaging and guidance to assist a physician. Additionally, the system may provide a physician with the ability to perform the procedure from an ergonomic position without the need for awkward arm motions and positions. Still further, the system may provide a physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart

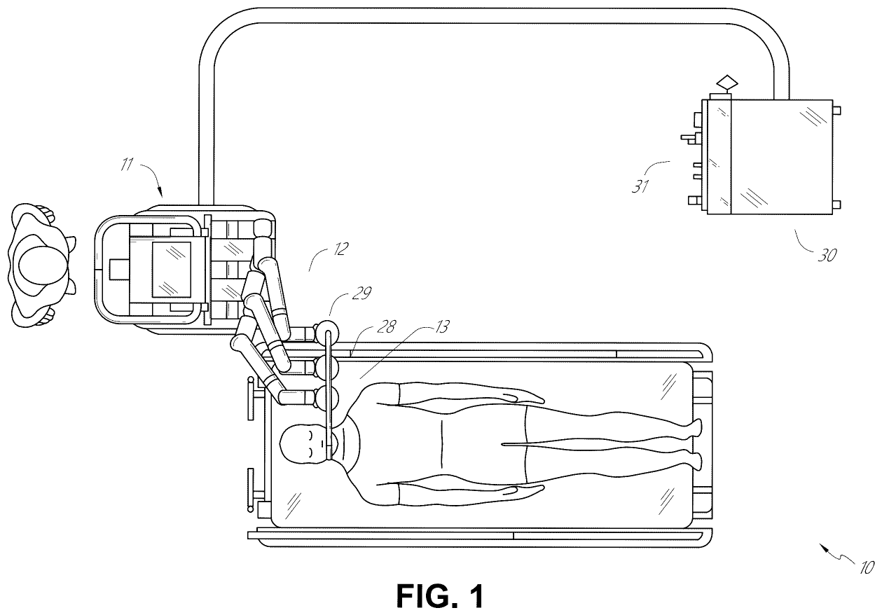

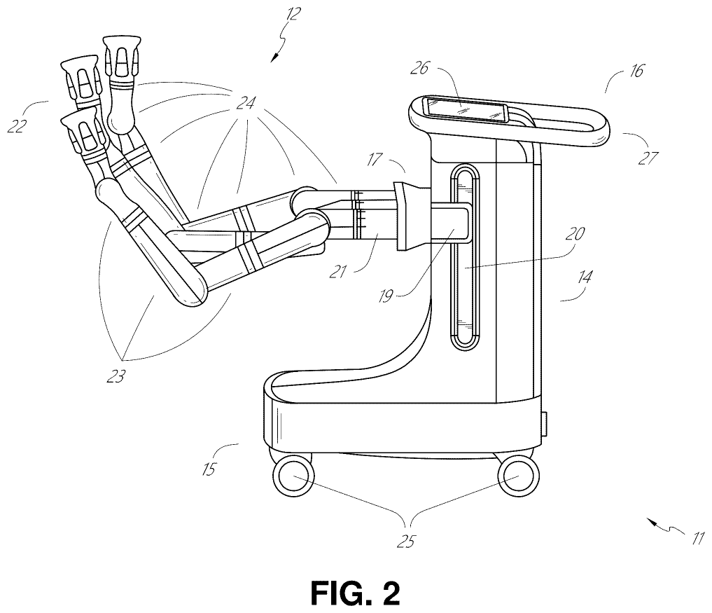

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independently of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart / table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of the tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include optoelectronics equipment for detecting, receiving, and processing data received from the optical sensors or cameras throughout the robotic system 10. In combination with the control system, such optoelectronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for a physician operator. Consoles in the system 10 are generally designed to provide both robotic controls as well as preoperative and real-time information of the procedure, such as navigational and localization information of the endoscope 13. When the console 31 is not the only console available to a physician, it may be used by a second operator, such as a nurse, to monitor the health or vitals of the patient and the operation of the system 10, as well as to provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart 11, the support for controls, optics, fluidics, and/or navigation may be provided through a separate cable.

FIG. 2 provides a detailed illustration of an embodiment of the cart 11 from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage 17 at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of the robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when the carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm 12. Each of the robotic arms 12 may have seven joints, and thus provide seven degrees of freedom. A multitude of joints result in a multitude of degrees of freedom, allowing for “redundant” degrees of freedom. Having redundant degrees of freedom allows the robotic arms 12 to position their respective end effectors 22 at a specific position, orientation, and trajectory in space using different linkage positions and joint angles. This allows for the system to position and direct a medical instrument from a desired point in space while allowing a physician to move the arm joints into a clinically advantageous position away from the patient to create greater access, while avoiding arm collisions.

The cart base 15 balances the weight of the column 14, carriage 17, and robotic arms 12 over the floor. Accordingly, the cart base 15 houses heavier components, such as electronics, motors, power supply, as well as components that either enable movement and/or immobilize the cart 11. For example, the cart base 15 includes rollable wheel-shaped casters 25 that allow for the cart 11 to easily move around the room prior to a procedure. After reaching the appropriate position, the casters 25 may be immobilized using wheel locks to hold the cart 11 in place during the procedure.

Positioned at the vertical end of the column 14, the console 16 allows for both a user interface for receiving user input and a display screen (or a dual-purpose device such as, for example, a touchscreen 26) to provide a physician user with both preoperative and intraoperative data. Potential preoperative data on the touchscreen 26 may include preoperative plans, navigation and mapping data derived from preoperative computerized tomography (CT) scans, and/or notes from preoperative patient interviews. Intraoperative data on display may include optical information provided from the tool, sensor and coordinate information from sensors, as well as vital patient statistics, such as respiration, heart rate, and/or pulse. The console 16 may be positioned and tilted to allow a physician to access the console 16 from the side of the column 14 opposite the carriage 17. From this position, a physician may view the console 16, robotic arms 12, and patient while operating the console 16 from behind the cart 11. As shown, the console 16 also includes a handle 27 to assist with maneuvering and stabilizing the cart 11.

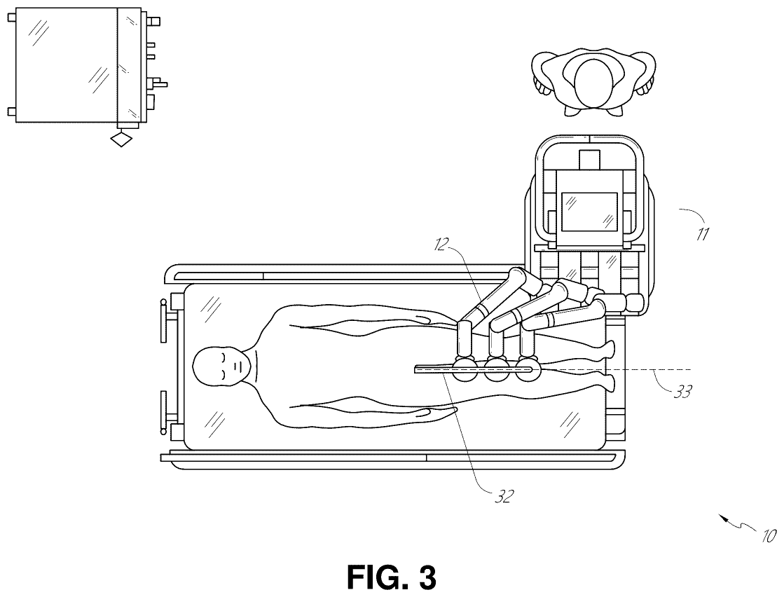

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

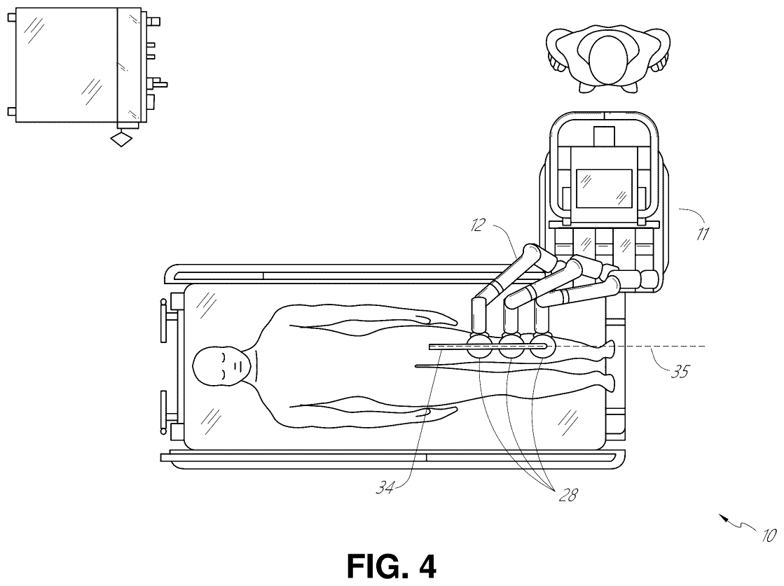

FIG. 4 illustrates an embodiment of a robotically-enabled system 10 similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table

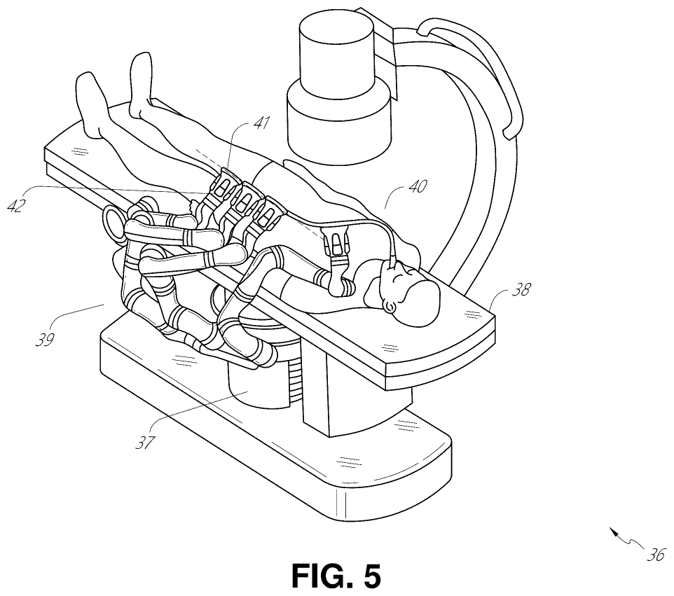

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopic procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around the table 38.

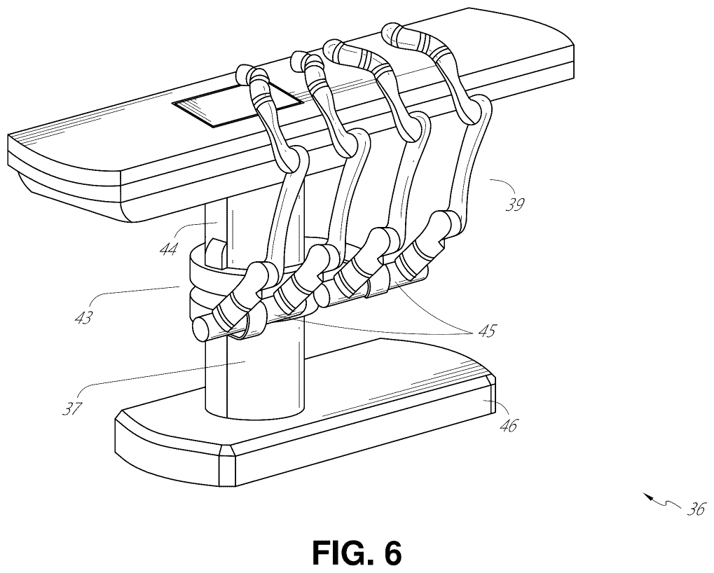

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independently of the other carriages. While the carriages 43 need not surround the column 37 or even be circular, the ring-shape as shown facilitates rotation of the carriages 43 around the column 37 while maintaining structural balance. Rotation and translation of the carriages 43 allows the system 36 to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

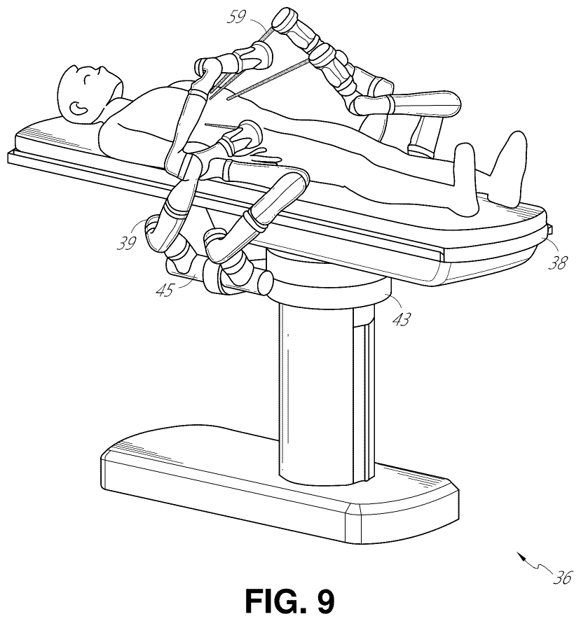

The robotic arms 39 may be mounted on the carriages 43 through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of the table 38 (as shown in FIG. 6), on opposite sides of the table 38 (as shown in FIG. 9), or on adjacent sides of the table 38 (not shown).

The column 37 structurally provides support for the table 38, and a path for vertical translation of the carriages 43. Internally, the column 37 may be equipped with lead screws for guiding vertical translation of the carriages, and motors to mechanize the translation of the carriages 43 based the lead screws. The column 37 may also convey power and control signals to the carriages 43 and the robotic arms 39 mounted thereon.

The table base 46 serves a similar function as the cart base 15 in the cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

With continued reference to FIG. 6, the system 36 may also include a tower (not shown) that divides the functionality of the system 36 between the table and the tower to reduce the form factor and bulk of the table. As in earlier disclosed embodiments, the tower may provide a variety of support functionalities to the table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base 46 for potential stowage of the robotic arms 39. The tower may also include a master controller or console that provides both a user interface for user input, such as keyboard and/or pendant, as well as a display screen (or touchscreen) for preoperative and intraoperative information, such as real-time imaging, navigation, and tracking information. In some embodiments, the tower may also contain holders for gas tanks to be used for insufflation.

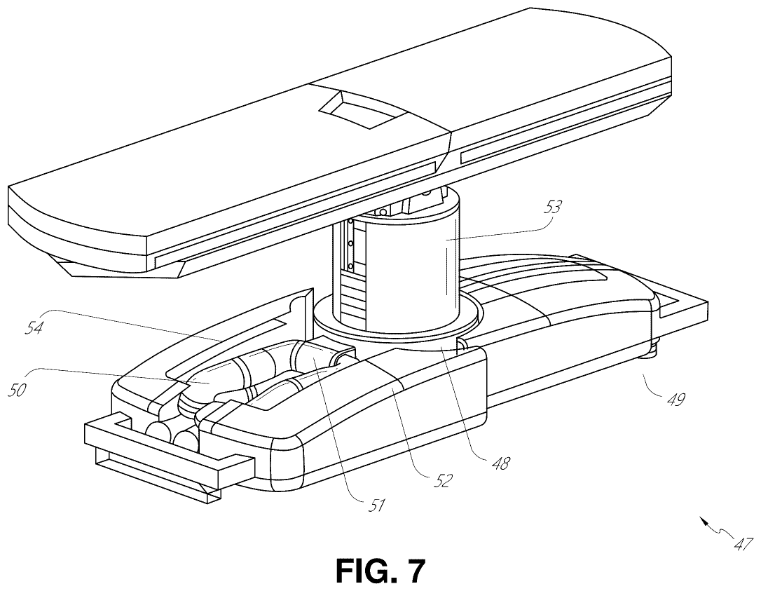

In some embodiments, a table base may stow and store the robotic arms when not in use. FIG. 7 illustrates a system 47 that stows robotic arms in an embodiment of the table-based system. In the system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and robotic arms 50 around column 53, and closed to stow to protect them when not in use. The base covers 52 may be sealed with a membrane 54 along the edges of its opening to prevent dirt and fluid ingress when closed.

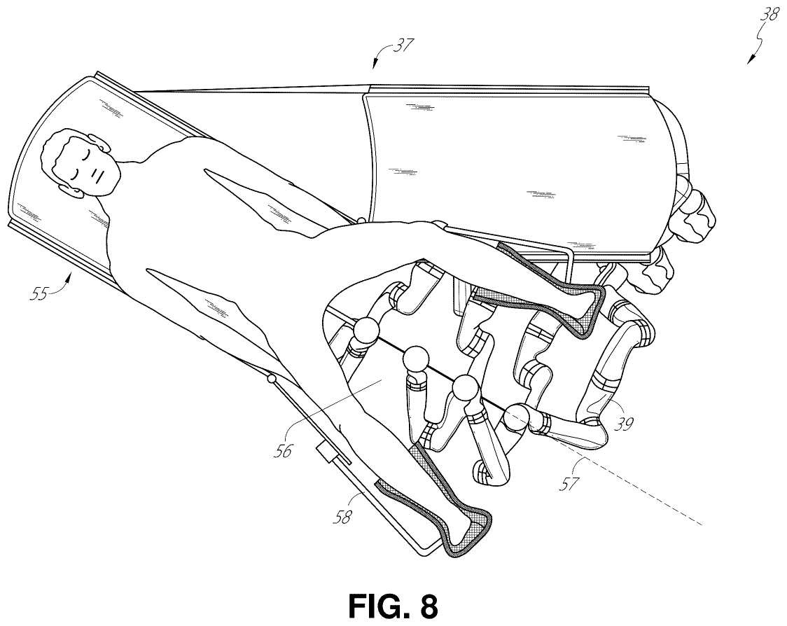

FIG. 8 illustrates an embodiment of a robotically-enabled table-based system configured for a ureteroscopic procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

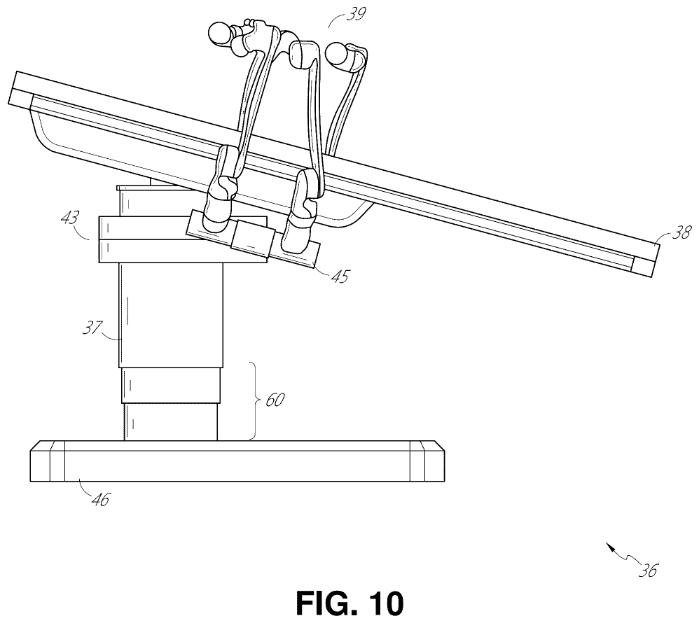

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the robotic arms 39 maintain the same planar relationship with the table 38. To accommodate steeper angles, the column 37 may also include telescoping portions 60 that allow vertical extension of the column 37 to keep the table 38 from touching the floor or colliding with the table base 46.

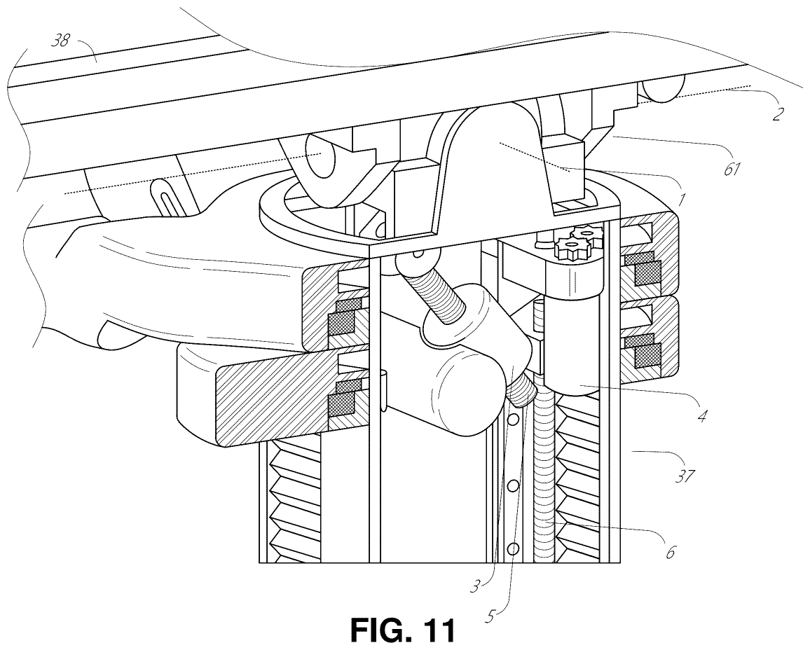

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's upper abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

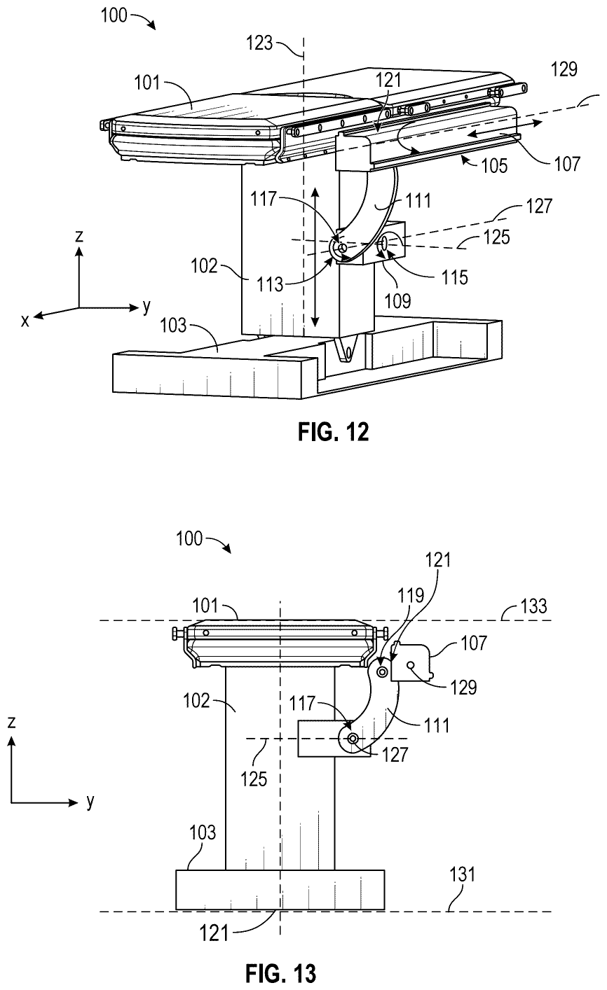

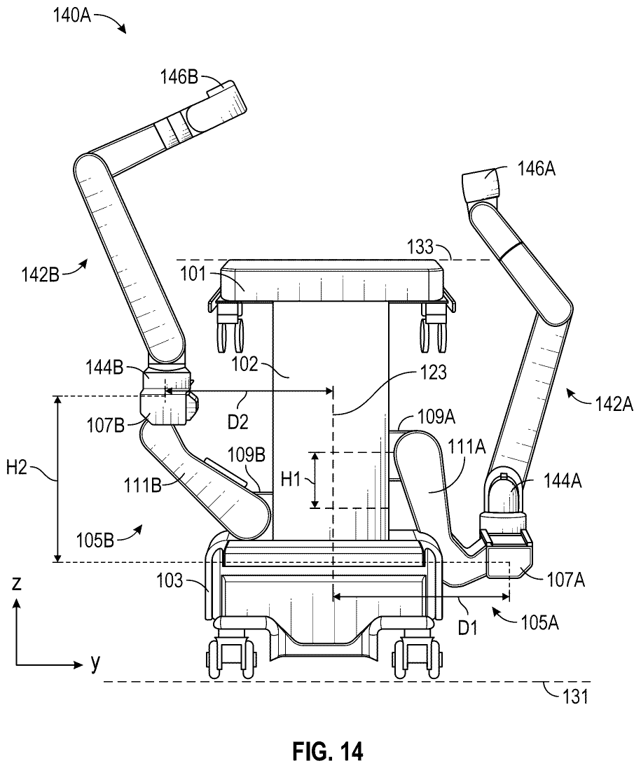

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface

The end effectors of the system's robotic arms may comprise (i) an instrument driver (alternatively referred to as “instrument drive mechanism” or “instrument device manipulator”) that incorporates electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by a physician or a physician's staff. In contrast, the instrument drivers need not be changed or sterilized, and may be draped for protection.

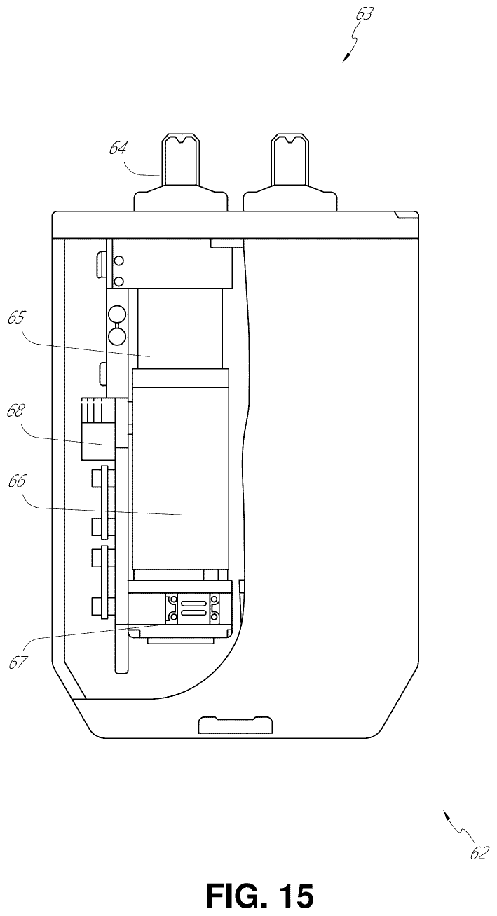

FIG. 15 illustrates an example instrument driver. Positioned at the distal end of a robotic arm, instrument driver 62 comprises one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuity 68 for receiving control signals and actuating the drive unit. Each drive unit 63 being independently controlled and motorized, the instrument driver 62 may provide multiple (e.g., four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument

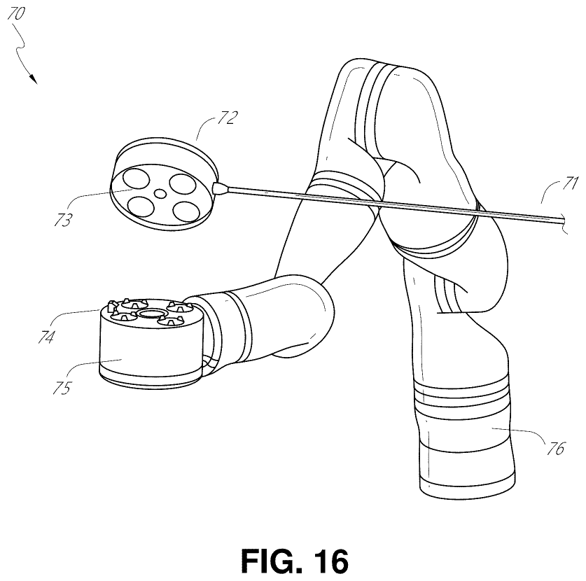

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by a physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of the instrument base 72 may share axes of rotation with the drive outputs 74 in the instrument driver 75 to allow the transfer of torque from the drive outputs 74 to the drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the elongated shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at the distal end of the elongated shaft 71, where tension from the tendon causes the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on the drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing therebetween may be altered or engineered for specific purposes, wherein tighter spiraling exhibits lesser shaft compression under load forces, while lower amounts of spiraling results in greater shaft compression under load forces, but limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft 71 may comprise a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include an optical camera. The shaft 71 may also accommodate optical fibers to carry light from proximally-located light sources, such as light emitting diodes, to the distal end of the shaft 71.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft 71. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft 71 during an endoscopic procedure.

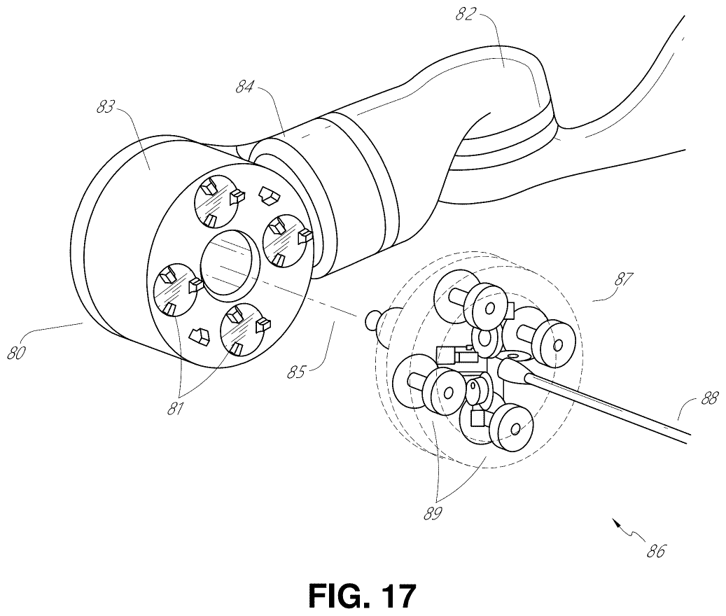

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver 80. Power and controls signals may be communicated from the non-rotational portion 84 of the instrument driver 80 to the rotational assembly 83 through electrical contacts that may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, the instrument shaft 88 extends from the center of the instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

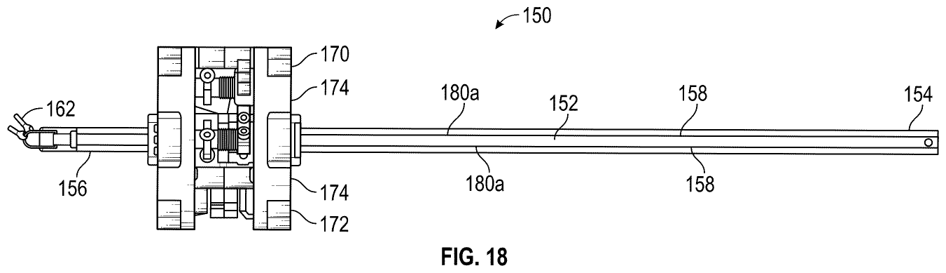

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

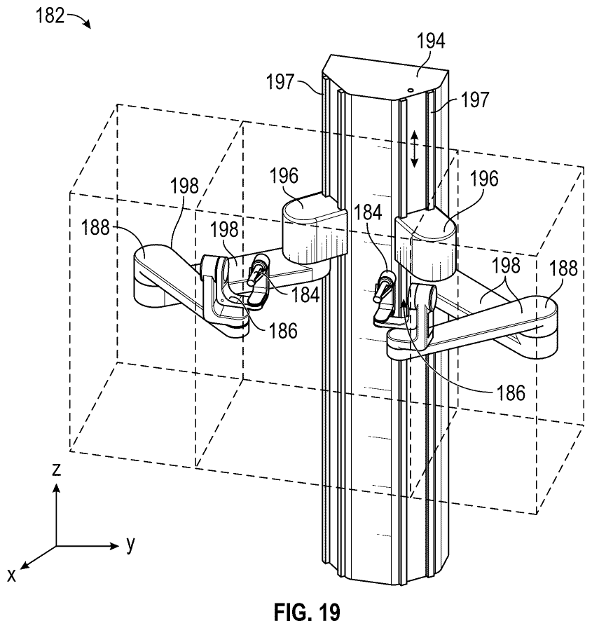

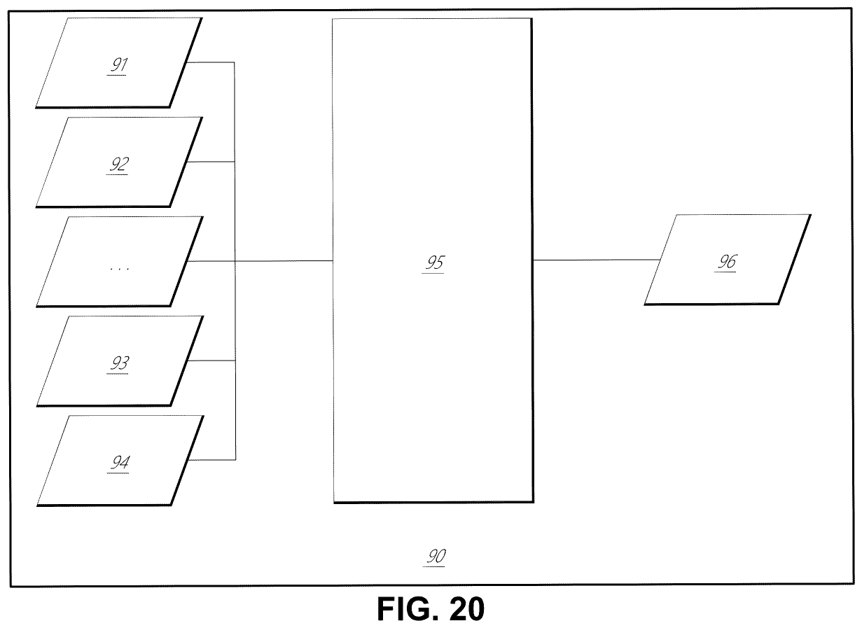

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as preoperative mapping, computer vision, real-time EM tracking, and robot command data may be used individually or in combination to achieve a radiation-free operating environment. In other cases, where radiation-based imaging modalities are still used, the preoperative mapping, computer vision, real-time EM tracking, and robot command data may be used individually or in combination to improve upon the information obtained solely through radiation-based imaging modalities.

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart 11 shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Preoperative mapping may be accomplished through the use of the collection of low dose CT scans. Preoperative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data (or image data) 92. The localization module 95 may process the vision data 92 to enable one or more vision-based (or image-based) location tracking modules or features. For example, the preoperative model data 91 may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intraoperatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intraoperatively “registered” to the patient anatomy (e.g., the preoperative model) in order to determine the geometric transformation that aligns a single location in the coordinate system with a position in the preoperative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during preoperative calibration. Intraoperatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Introduction to Robotic Tool Movement

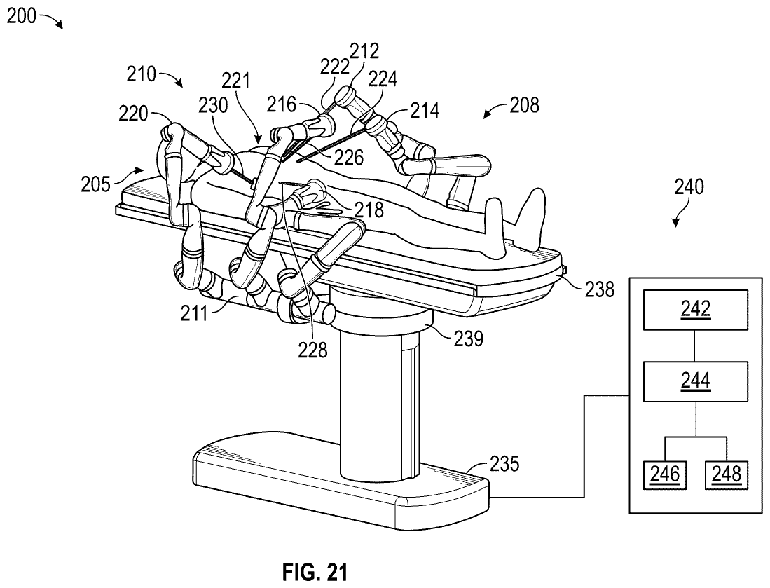

The present disclosure is directed to the coordinated or synchronized movement between two or more robotically controlled tools (e.g., one or more instruments and/or cameras). FIG. 21 illustrates a robotic medical system 200. As shown in several of the examples above, robotic medical systems can be used for medical procedures, such as, e.g., endoscopy, laparoscopy, or others (see, for example, FIGS. 1, 3-5, 8, and 9, described above). The robotic medical system 200 can include a patient side platform 210. The patient side platform 210 can include a patient table 238. The patient table 238 can be sized to support a patient 205 during the medical procedures. The patient table 238 can be movable and/or include one or more movable sections to manipulate a position of the patient 205. The patient table 238 can be supported by a column 239 and a table base 235.

The patient side platform 210 can include at least one or more robotic arms 208. The robotic arms 208 can be mounted on one or more rails 211. The rails 211 can be movably mounted to the patient table 238. Not all of the robotic arms 208 need be utilized for each medical procedure. Accordingly, unused robotic arms 208 can be stowed or detached from the patient side platform 210.

The robotic arms 208 can include a first robotic arm 212, a second robotic arm 214, a third robotic arm 216, a fourth robotic arm 218, and/or a fifth robotic arm 220. Though not illustrated, one or more additional robotic arms 208 can also be provided. The robotic arms 208 may each generally comprise robotic arm bases and end effectors separated by a series of linkages that are connected by a series of joints. The joints can include independent actuators having independently controllable motors for maneuvering the robotic arms 212-220. In certain implementations, the robotic arms 208 can include seven movable joints.

The robotic arms 208 can include or be coupled to one or more robotic tools 221 for performing robotically controlled medical procedures, such as robotic surgery. The robotic tools 221 can comprise one or more instruments and/or cameras. The instruments can comprise, but are not limited to, monopolar shears, needle drivers, monopolar hooks, tissue graspers, vessel sealers, and/or staplers. For example, the instruments can be rigid (e.g., laparoscopic) instruments. In one embodiment, one of the instruments can comprise a rigid laparoscope with one or more cameras. The robotic tools 221 can include a first instrument 222, a second instrument 224, a third instrument 226, a fourth instrument 228, and/or a camera 230. The robotic tools 221 can each correspond to a respective arm of the robotic arms 208. The robotic tools 221 can be inserted through incisions or natural orifices into a patient's body, such as within the patient's abdomen, and navigated to a worksite for performing medical procedure(s). The respective robotic arms 208 can control positions of the robotic tools 221 during robotically controlled medical procedures.

To control the patient side platform 210, including the robotic arms 208 and robotic tools 221, the robotic medical system 200 can include a controller 240. FIG. 21 illustrates a schematic embodiment of the controller 240. The controller 240 can include a viewer 242. The viewer 242 can include any combination of graphical user interfaces, screens, projections, stereoscopic viewers, or other visual interfaces, controller inputs, haptic feedback systems, etc. for a physician to operate the patient side platform 210. The viewer 242 can also include a master controller 244. The master controller 244 can include one or more controllers (e.g., multiple degree-of-freedom gimbals, keypads, mouse, etc.) for operating the robotic arms 208 and the robotic tools 221 of the patient side platform 210. In certain implementations, the controller 240 can be a physician's console.

The controller 240 can include a processor 248. The processor 248 can receive input signals from the master controller 244 to control positions of the robotic tools 221 and robotic arms 208. The processor 248 can receive input signals from the patient side platform 210 indicating positions of the robotic tools 221 and/or the robotic arms 208 (e.g., from position encoders mounted on the robotic arm joints).

The processor 248 can be communicatively coupled with a computer readable storage medium 246. The computer readable storage medium 246 can have stored thereon instructions executable by the processor 248. The instructions can cause the processor 248 to output signals for moving the robotic arms 208 and/or robotic tools 221 based on the input signals. The instructions on the computer readable storage medium 246 can include virtual models of the patient side platform 210. The virtual models can represent (e.g., mathematically) the positions of the robotic tools 221 and robotic arms 208. The virtual model can be formed, at least in part, by the input data from the robotic tools 221 and/or robotic arms 208. Accordingly, the master controller 244 can control the robotic arms 208 and/or robotic tools 221 in a master-slave relationship.

While the embodiment in FIG. 21 illustrates robotic arms 208 that are coupled to a patient side platform 210 that is akin to a bed, in other embodiments, the robotic arms 208 can be coupled to a patient side platform that is akin to a cart, as shown in FIGS. 1-4. One skilled in the art will appreciate that the next sections that describe coordinated movement of robotic tools can be applicable to tools that are coupled to robotic arms on either a bed or a cart.

### 3. Coordinated Robotic Tool Movement

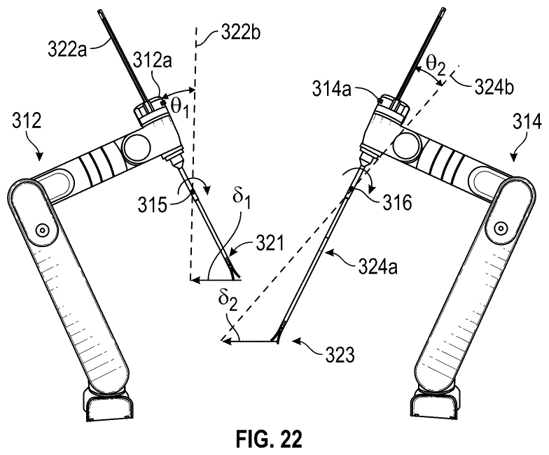

FIG. 22 illustrates a first robotic arm 312 and a second robotic arm 314. The robotic arm 312 can be an implementation of one of the robotic arms 208. The robotic arm 312 can include or be coupled to a first instrument 322. The instrument 322 can be coupled on an end effector 312a of the robotic arm 312 (e.g., on the last joint of the robotic arm 312). The instrument 322 can be extendable and retractable with respect to the end effector 312a. The instrument 322 can also be articulable in space by movement of the linkages of the robotic arm 312. The instrument 322 can be moved from a first pose 322a to a second pose 322b. The movement of the instrument 322 can include a change of angle θ₁ as shown. The movement of the instrument 322 can include change of position of an instrument distal end 321. The distal end 321 can move along a path δ₁. The path δ₁ can extend in three dimensions. The movement of the instrument 322 can include an extension and/or retraction of the instrument 322 within the end effector 312a. In certain implementations, the path δ₁ can be measured at the distal end 321, or any location along the instrument 322.

As the instrument 322 moves from the first pose 322a (i.e., a first position and first orientation) to the second pose 322b (i.e., the distal end 321 moves along the path δ₁), the instrument 322 can move about a remote center of motion 315. The remote center of motion 315 can be fixed in space (e.g., relative to the patient side platform 210). In some embodiments, maintaining the remote center of motion 315 during movement of the instrument 322 and robotic arm 312 can be automated.

According to one automated implementation of the robotic arm 312, a user can control a master controller (not shown) to cause the distal end 321 to move along the path δ₁. An input signal from the master controller can be translated into a virtual path δ₁′ within a virtual model of a virtual robotic arm 312′ and a virtual robotic instrument 322′. Translation of the path δ₁ into the virtual path δ₁′ can include a scaling factor, such as 3:1, 4:1, etc. The requisite movements of the virtual robotic arm 312′ and/or virtual robotic instrument 322′ that are required to move the virtual robotic instrument 322′ along the virtual path δ₁′ can be calculated using reverse kinematics. The reverse kinematic calculation can include a virtual center of motion 315′ of the virtual robotic instrument 322′ as a limitation of the virtual model.

The requisite movements of the virtual model calculated using the reverse kinematics can be translated into corresponding physical movements of the robotic arm 312 and/or the instrument 322. Servos, motors or other actuators on the robotic arm 312 and/or the instrument 322 can move the distal end 321 to move along the path δ₁, in accordance with the calculated requisite movements from the virtual model. The arm 312 can adjust to move instrument 322 through the angle θ₁. The end effector 312a can adjust the length of the instrument 322. The remote center of motion 315 can remain in place during the executed movement.

The second robotic arm 314 can be structurally and/or functionally similar to, or the same as, the first robotic arm 312. The robotic arm 314 can include a second instrument 324. The instrument 324 can be coupled on an end effector 314a of the robotic arm 314. The instrument 324 can be moved from a first pose 324a to a second pose 324b. The movement of the instrument 324 can include a change of angle θ₂ and/or a movement of a distal end 323 along a path δ₂. Movement of the instrument 324 along the path δ₂ can be in response to user input at the master control. Movement of the robotic arm 314 about a center of motion 316 can be automated as the instrument 324 moves along the path δ₂, as described above for the instrument 322.

One aspect of the disclosure is coordinated movement between the first and second instruments 322, 324. A user can control the first instrument 322 to move along the path δ The second instrument 324 can be automatically controlled to move with the first instrument 322 to maintain a relative spacing between the first and second instruments 322, 324. For example, the relative spacing can be maintained between the distal ends 321, 323. In certain implementations, the path δ₂ can parallel (i.e., track) the path δ₁ to maintain the relative spacing. Movement of the second instrument 324 can be concurrent with movement of the first instrument 322.

In certain implementations, additional robotic instruments can be automatically controlled to move with the first instrument 322. Movements of the additional instruments can maintain relative spacings with the first instrument 322. Movements of the additional instruments can be concurrent with the movement of the first instruments 322 along the path δ₁. Accordingly, multiple robotic instruments (e.g., two or more) can be moved in a coordinated manner.

### 4. Coordinated Robotic Tool Movement While Maintaining Remote Centers of Motion Located at Multiple Ports

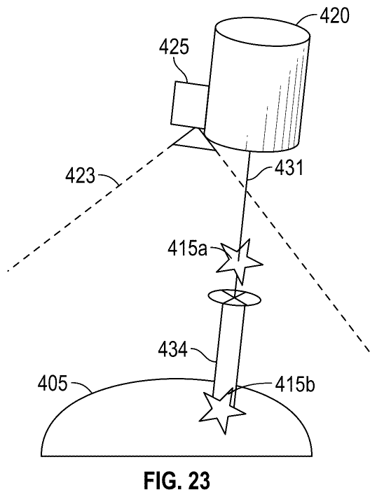

In general, remote centers of motion can be important for patient safety during laparoscopic, thoracic, percutaneous and other medical procedures. FIG. 23 schematically illustrates an instrument 431 connected with the end of a robotic arm 420. The robotic arm 420 can include a camera 425. The instrument 431 can move about a remote center of motion 415a. The instrument 431 can be inserted into a body 405 of a patient through a port or cannula 434 (e.g., trocar). The cannula 434 can extend through an incision or natural orifice in the body 405.

The instrument 431 can be advanced into the cannula 434 such that the remote center of motion 415a can be at or associated with an interface of the patient's body 405 and the instrument 431 or cannula 434 (e.g., at 415b). This positioning of the remote center of motion 415a can allow movement of the distal end of the instrument 431 within the body 405 while preventing damage done to the body 405 (e.g., at the interface) by the movement of the shaft of the instrument 431.

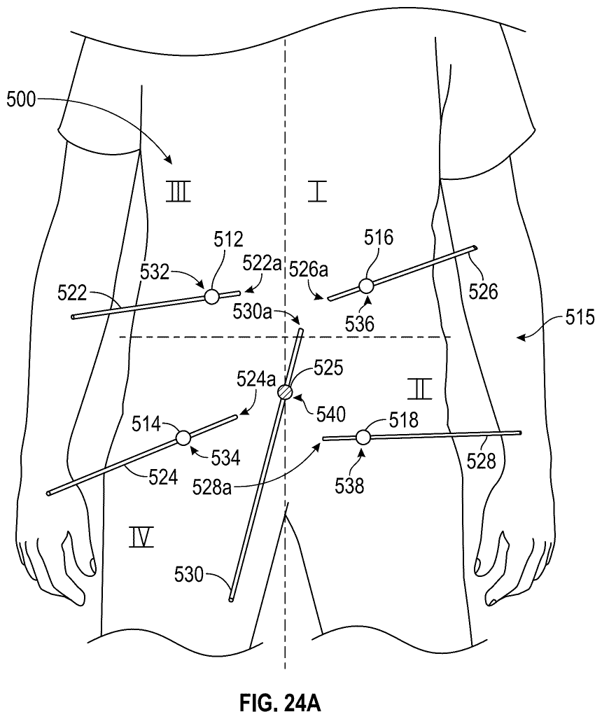

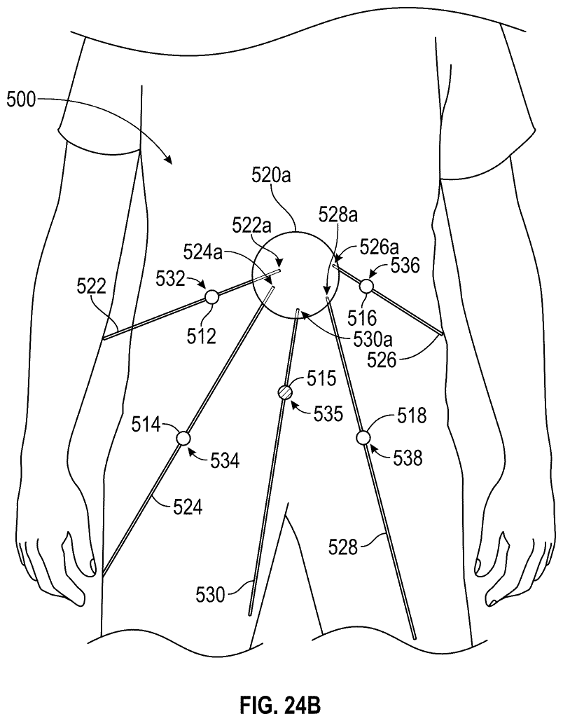

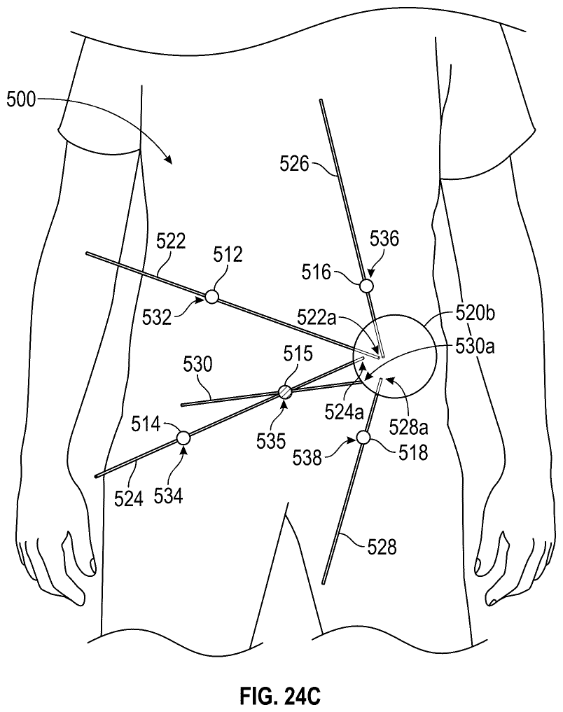

Some medical procedures involve access to more than one workspace within the patient's body during the same medical episode. For example, with reference to the implementation of FIG. 24A, during a total colectomy, a physician may need to perform surgical tasks in the four quadrants (e.g., right upper quadrant I, right lower quadrant II, left upper quadrant III, left lower quadrant IV) of an abdomen 500. This requires movement of two or more instruments from worksite to worksite.

Known robotic medical systems do not allow control of instruments and a camera simultaneously through different ports. Accordingly, to move all three robotic tools from worksite to worksite, a user would have to make several small movements, alternating control between the instruments and the camera (e.g., to keep instruments in field of view of the camera). If a user has a third instrument in the body, the process of moving from worksite to worksite is even more onerous. A user would need to move the first two instruments to the new worksite making the several small movements and switching between camera and instrument control and then move the camera back to the original worksite. The user can then swap control to the third instrument and bring the third instrument to the new worksite. This sequence typically results in instruments being left off-screen, which is a hazardous scenario for the patient.

In addition, safe movement of any robotic instrument may require that the view provided by the camera be directionally oriented with the controls. This may require an orientation process whereby the controls and the camera view are aligned. However, movement of the camera can disorient the user and generally requires reorientation of the camera with the controls. Accordingly, each time the user moves the camera, switches back and forth between the camera and the instruments, or leaves the instruments off screen and returns thereto can require reorientation of the user controls or camera. This process can be time consuming and inconvenient.

In addition, some medical procedures are conducted with robotically controlled instruments inserted through multiple ports. For example, during laparoscopic surgery, the patient's abdomen can include a plurality of ports through which one or more robotic tools can be inserted. Each of the ports can include an incision through the abdominal wall and/or a cannula, as described above.

With continued reference to the implementation of FIG. 24A, the ports can include a first port 512, a second port 514, a third port 516, a fourth port 518 and/or a fifth port 525. Although not required, the ports 512, 514, 516, 518 can be located within any or all of the four quadrants (I, II, III, IV) of the abdomen 500.

The robotic tools 515 can include a first instrument 522, a second instrument 524, a third instrument 526, a fourth instrument 528, and/or a camera (e.g., laparoscope) 530. The first instrument 522 can be inserted into the abdomen 500 through the first port 512. The first instrument 522 can include a remote center of motion 532. The remote center of motion 532 can be at the port 512. The first instrument 522 can include a distal end 522a. Similar to the first instrument 522, the second, third, fourth instruments 524-528 and/or camera 530 can include respective remote centers of motion 534-538, respective distal ends 524a-530a, and be inserted into the abdomen 500 through the respective ports 514-518.

The systems and methods disclosed herein allow coordinated motion between robotic tools (e.g., instruments and/or cameras). These robotic tools can also maintain remote centers of motion during such coordinated movement. FIG. 24A shows the patient's abdomen 500 undergoing laparoscopic surgery as an exemplary application environment for the systems and methods disclosed herein. FIG. 24B-C illustrate coordinated movement of robotic tools between worksites 520a, 520b. In FIG. 24B, the robotic tools 515 can be located at a first worksite 520a. In FIG. 24C, the robotic tools 515 have been moved to a second worksite 520b. In certain implementation, the movements of the robotic tools 515 can be coordinated as they are moved from the first worksite 520a to the second worksite 520b.

Each of the robotic tools 515 can include a remote center of motion. The remote centers of motion can be located at the respective ports 512-518 (e.g., at the intersection of the robotic tools 515 with the abdominal wall of the abdomen 500). The remote centers of motion can be maintained as the robotic tools 515 are moved by respective robotic arms (not shown), including during coordinated movement of the robotic tools 515.

FIG. 24B shows the distal end 522a of the first instrument 522 located at the first worksite 520a. A robotic arm (not shown) can position the distal end 522a within the worksite 520a. The remote center of motion 532 can remain at the port 512. FIG. 24C shows the distal end 522a of the first instrument 522 moved to the second worksite 520b. A robotic arm (not shown) can move the distal end 522a from worksite 520a to worksite 520b while maintaining the remote center of motion 532 at the port 512. Similarly, the distal ends 524a-530a of the second, third, fourth instruments 524-528 and/or camera 530 can be moved to the second worksite 520b from the first worksite 520a within the abdomen 500. Respective robotic arms (not shown) can move the distal ends 524a-530a while maintaining the remote centers of motion 534-538 at the respective ports 514-518.

Movement of the instruments 522-528 and/or camera 530 from worksite 520a to worksite 520b can be onerous and time consuming if done individually. One aspect of the present disclosure is coordinated movement of two or more of the instruments 522-528 and/or camera 530.

According to a follower mode, the poses of the distal ends 522a-528a can be coordinated with the pose of the distal end 530a of the camera 530. A user can control the pose of the camera 530 using a controller (e.g., a single controller). Movement of the camera 530 can cause a coordinated movement of the instruments 522-524 (or any subset thereof). The distal ends 522a-528a can maintain a relative spacing with the distal end 530a of the camera 530. During this coordinated movement, the remote centers of motion 532-538 of the instruments 522-528 and camera 530 can automatically remain at the respective ports 512-518. By moving the camera 530 from the first worksite 520a to the second worksite 520b, the instruments 522-528 can be automatically moved from the first worksite 520a to the second worksite 520b.

According to a camera mode, a variation of the follower mode, the positions of the distal ends 522a-528a can be coordinated with field of vision of the camera 530. In FIG. 24B, the camera 530 has a particular orientation in which the first worksite 520a and/or each of the distal ends 522a-528a are within the field of view of the camera 530. A user can control the field of view of the camera 530 using the controller. Movement of the field of view of the camera 530 can cause a coordinated movement of the instruments 522-524 (or any subset thereof). The instruments 522-524 can be moved while maintaining the distal ends 522a-528a within the field of vision of the camera 530. During this coordinated movement, the remote centers of motion 532-538 of the instruments 522-528 and camera 530 can automatically remain at the respective ports 512-518. In FIG. 24C, the camera 530 is moved to view the second worksite and/or the each of the distal ends 522a-528a remain within the field of view of the camera 530.

According to a dual control mode, movement of the instruments 522-524 (or any subset thereof) can be controlled using a first controller. The camera 530 can be controlled using a second controller. The first and second controllers can be independently operated by a user. During coordinated movement of the instruments 522-524 by the first controller, the remote centers of motion 532-538 of the instruments 522-528 can automatically remain at the respective ports 512-518. In this implementations, a user can navigate the instruments 522-524 together in coordinated movements along a pathway from the first worksite 520a to the second worksite 520b by operation of the first controller. The user can also navigate the camera 530 along a pathway from the first worksite 520a to the second worksite 520b by operation of the second controller. Desirably, the user can maintain the instruments 522-524 within the field of view of the camera 530 during this movement. Alternatively, movement of the field of view of the camera 530 using the second controller can cause automatic coordinated movement of the instruments 522-524 (or any subset thereof) to stay within the field of vision. The first controller can be used to move or adjust the instruments 522-524 (or any subset thereof) within the field of vision.

The coordinated movement of the instruments 522-528 and/or camera 530 can reduce the total time required to move from the first worksite 520a to the second worksite 520b. The coordinated movement of the instruments 522-528 and/or camera 530 can also reduce risks associated with leaving one or more instruments within the abdomen 500 while outside the field of view of the camera 530. Moreover, the orientation of the camera 530 can be maintained relative to the instruments 522-528.

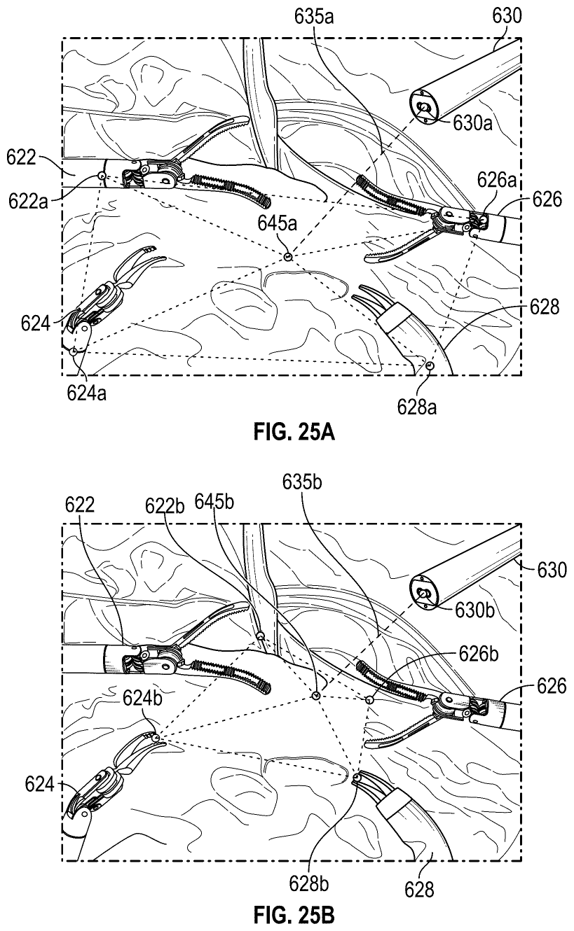

FIGS. 25A-B illustrate instruments 622, 624, 626, 628 and camera 630 of a robotic medical system at a worksite 620a. In some implementations, the instruments 622-628 can include gripper and/or cutter type instruments, although any instrument type is contemplated herein. Movements of the instruments 622-628 (e.g., from or to the worksite 620a) can be coordinated with the movement of the camera 630, as described above. Various methods can be used for coordinating the relative positioning of the instruments 622-628 and the camera 630.

FIG. 25A illustrates a first relative spacing 635a of the instruments 622-628 and the camera 630. The instruments 622-628 can include respective reference points 622a, 624a, 626a, and 628a respectively. The reference points 622a-628a can be any location on the instruments, 622-628. The reference points 622a-628a can be at the respective distal ends of the instruments 622-628. The camera 630 can include a reference point 630a. The reference points 622a-630a can be linked together by one or more lines (e.g., diagonals). The lines can form a crossing point 645a. The crossing point 645a can be generally at a center location of the instruments 622-628 (or any subset thereof). The reference point 630a can be at a distal end of the camera 630 (e.g., at a lens of the camera 630). The reference points 622a-630a can be used to define the relative spacing 635a between the camera 630 and any or all of the instruments 622-628. The relative spacing 635a can be defined between the crossing point 645a and the reference point 630a. In the follower mode, the relative spacing 635a can be maintained. In the camera mode, the field of vision of the camera 630 can be directed towards (e.g., centered on) the crossing point 645a. In the dual control mode, the relative spacing of the instruments 622-628 about the crossing point 645a is maintained.

FIG. 25B illustrates a second relative spacing 635b of the instruments 622-628 and the camera 630. The instruments 622-628 can include reference points 622b-628b, respectively. The reference points 622b-628b can be located at gripping or cutting points for the instruments 622-628 (e.g., midway between the jaws of the instruments). The reference points 622b-628b can be linked together by one or more lines. The lines can form a crossing point 645b. The crossing point 645b can be generally at a center location of the instruments 622-628 (or any subset thereof). The camera 630 can include a reference point 630b. The relative spacing 635b can be defined between the crossing point 645b and the reference point 630b. In the follower mode, the relative spacing 635b can be maintained. In the camera mode, the field of vision of the camera 630 can be directed towards (e.g., centered on) the crossing point 645b. In the dual control mode, the instruments 622-628 can maintain relative spacings about the crossing point 645b.

For any of the above systems and operation modes, certain circumstances may occur that cause one or more of the robotic arms to be unable to follow along the synchronous motions or paths, or unable to maintain the relative spacings between robotic arms and/or instruments attached to the robotic arms. For example, the robotic arms can reach motion limits, encounter obstructions, interfere with one another or surrounding objects, etc. Accordingly, the system can have pre-determined responses to these circumstances. In one implementation, the system can arrest or disable the motion of all the robotic arms and/or movement of the instruments by instrument device manipulators of the robotic arms. In another implementation, the system can arrest only the robotic arm (and/or the instrument device manipulator of the robotic arm) that is unable to meet the motion commitment or request. In another implementations, the system can include an audible, visual, haptic, or other alert that indicates to a user that the one or more components of the robotic system (e.g., arms) are unable to meet the motion commitment. In another implementation, the system use any combination of the above response strategies.

### 5. Controls for Coordinated Movement of Robotic Tools

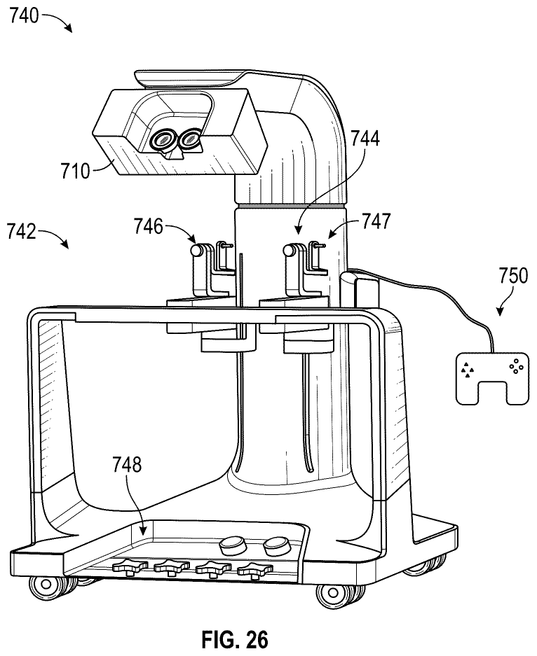

FIG. 26 shows a controller 740 for controlling movement of a plurality of robotic arm and/or robotic tools, such as those described in the robotic medical systems described herein. The controller 740 can be an implementation of the controller 240. The controller 740 can be a console. The controller 740 can include a user interface 742. The user interface 742 can include a viewer 710. The viewer 710 can be a screen, projection, stereoscopic viewer, or other visual interface. The viewer 710 can display images or video taken through the a camera (e.g., robotic laparoscope) used during a medical procedure.

The controller 740 can include a master control 744. The master control 744 can include controls for robotic arms and/or the robotic tools. To control the robotic arms, a surgeon can be seated at the controller 740 (e.g., physician console) and will drive one or more of the robotic arms using the master controller 744. The controls can include a left gimbal 746 and/or a right gimbal 747. The left gimbal 746 can control a first robotic arm and a first robotic tool in a master-slave arrangement. The right gimbal 747 can control a second robotic arm and a second robotic tool in a master-slave arrangement.

The master control 744 can allow switching control between the robotic tools. In one implementations, the robotic medical system includes more than two robotic tools. The controller 740 can include one or more footpedals 748 (biased or unbiased), touchscreens, button or other switches. The footpedals 748 or other switches can enable a user to switch control between robotic arms/tools. The footpedals 748 or other switches can be used to assign control of the robotic tools to the left gimbal 746 or the right gimbal 747. Accordingly, fewer gimbals are required for controlling the patient side platform 210 than the number of robotic arms and instruments/cameras. In certain implementations, the footpedals 748 or other switches can initiate coordinated movement of the robotic tools. In certain implementations, the footpedals 748 or other switches can select the control mode (e.g., follower mode, camera mode, dual control mode, or other). In certain implementations, the coordinated movement can only be initiated under predicate conditions, such as all of the selected tools are within the view of the camera and/or all tools have been assigned to a controller (e.g., gimbals 746, 747).

The master control 744 can be used for the coordinated movements of the robotic tools described above. In the follower mode or the camera mode, the robotic tools (or a subset thereof) can be controlled using either the left gimbal 746 or the right gimbal 747. The left gimbal 746 can control the first robotic tool (e.g., robotic camera). The second robotic tool (e.g., an instrument) and/or other robotic tools can be moved in automated coordinated movements with the first robotic tool while maintaining respective remote centers of motion at respective ports.

In the dual control mode, the robotic tools (or a subset thereof) can be controlled using both the left gimbal 746 and the right gimbal 747. The left gimbal 746 can control the first robotic tool (e.g., robotic camera). The right gimbal 747 can control the second robotic tool (e.g., an instrument) and one or more other robotic tools. The second robotic tool and the one or more other robotic tools can be moved in automated coordinated movements while maintaining respective remote centers of motion at respective ports.

In certain implementations, the left gimbal 746 and the right gimbal 747 of the master control 744 are used to operate the robotic tools to perform robotic medical procedures at a worksite. In other implementations, the master control 744 includes a relocation controller 750 to move the robotic tools between worksites. The relocation controller 750 can include control inputs for selecting the robotic arms for coordinated movement. The relocation controller 750 can allow a user to select all or a subset of the robotic instruments. The relocation controller 750 can select to move the first robotic tool. The relocation controller 750 can select to move the second robotic tool in a coordinated movement with the first robotic tool. The relocation controller 750 can include a first controller to move the first robotic tool and a second controller to move the second robotic tool an one or more additional robotic tools in a coordinated movement with the second robotic tool. The relocation controller 750 or the controller 740 can allow selection of the control mode for the coordinated movements (e.g., follower, camera, dual, or other).

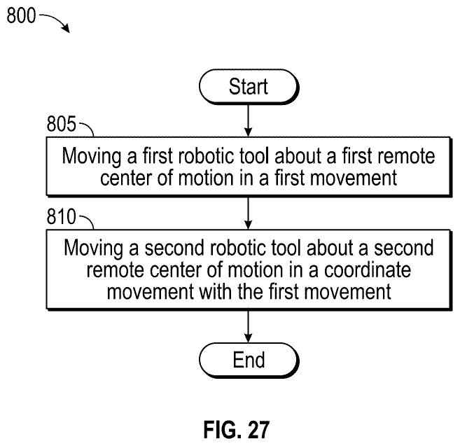

FIG. 27 shows a method 800 for performing a medical procedure using a robotic medical system. The robotic medical system can include a plurality of robotic arms. The plurality of robotic arms can include first and second robotic arms and corresponding first and second robotic tools. The robotic medical system can include a control unit configured to control movement of each of the robotic arms. The method 800 can start at block 801. The method 800 can be initiated by a user of the robotic medical system. Initiation can include enabling a coordinated movement mode of the robotic medical system and/or assignment of the plurality of robotic arms to one or more controls of the control unit.

At step 805, the user can operate the first robotic arm to move the first robotic tool in a first movement. The first robotic arm can maintain a first remote center of motion during the first movement. At step 810, the robotic medical system can automatically operate the second robotic arm to move the second robotic tool in a coordinated movement with the first movement. The second robotic arm can maintain a second remote center of motion during the coordinated movement. The coordinated movement can be simultaneous with the first movement or delayed. The method 800 ends at block 820.

### 6. Concomitant Procedures

The treatment of certain medical conditions may involve performing two or more medical procedures to fully treat the medical condition. For example, the diagnosis and management of pulmonary lesions may involve multiple treatment episodes to perform medical procedures including flexible endoscopy and thoracoscopy. Treatment of such conditions can be staged across multiple treatment episodes. However, staging treatments can increase risk and inconvenience to patients and increase perioperative resources leading to increased time and costs to both the patient and the physician.

Alternatively multiple treatment procedures can be performed serially or in parallel during single treatment episode. However, as for multiple treatment episodes, there are drawbacks associated with single treatment episodes as they are currently performed. As noted above, multiple clinical providers may need to assist in performing a single treatment episode, thereby leading to increased costs and an overcrowded space in the operating room. Furthermore, to perform multiple procedures serially over a single treatment episode, the physician may alternate between the various approaches, which may involve switching between sterile and non-sterile techniques. Switching between sterile and non-sterile techniques may further involve changing attention from one surgical site to another, regowning, and significantly interrupted clinical workflow.

Overall, the coordination of multiple healthcare providers and/or physicians to perform procedures in parallel during a single treatment episode is expensive and may be cost prohibitive for certain procedures. Accordingly, embodiments of the disclosure relate to systems and methods for performing two or more types/modes of procedures concomitantly (e.g., by a single user or team) as part of a single treatment episode. Robotic medical systems can be used to perform concomitant procedures, as further described in U.S. patent application Ser. No. 16/559310, filed Sep. 3, 2019, the entirety of which is hereby incorporated by reference.

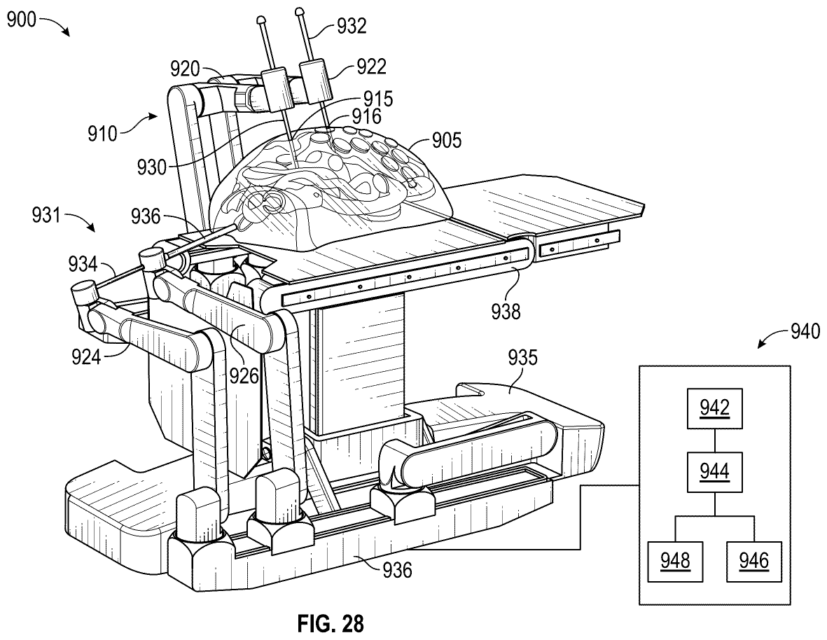

As show in FIG. 28, a robotic medical system 900 can be used to perform concomitant procedures on a patient 905. The robotic medical system 900 can include a patient bed 938 for supporting and positioning the patient 905. The robotic medical system 900 can include a base 935 for supporting the patient bed 938. The robotic medical system 900 can include a plurality of robotic arms 910. A first robotic arm 920 of the plurality of robotic arms can be used for controlling a first robotic tool 930. The first robotic tool 930 can be a rigid (e.g., laparoscopic) instrument. A distal end of the first robotic tool 930 can be inserted into the patient's body 905 through an incision (e.g., within an abdominal wall) and/or cannula. The first robotic tool 930 can rotate about a remote center of motion 915. The remote center of motion 915 can be located at the incision.

A second robotic arm 922 of the plurality of robotic arms 910 can be used for controlling a second robotic tool 932. The second robotic tool 932 can be a rigid (e.g., laparoscopic) instrument. A distal end of the second robotic tool 932 can be inserted into the patient's body 905 through an incision (e.g., within an abdominal wall) and/or cannula. The second robotic tool 932 can rotate about a remote center of motion 916. The remote center of motion 916 can be located at the incision.

The first and second robotic tools 930, 932 can be operable by a controller 940. The controller 940 can include a viewer 942. The controller 940 can include a master controller 944. The master controller 944 can be a console. The master controller 944 can be coupled with a processor 948. The processor 948 can receive input signals from the viewer 942 to control positions of first and second robotic tools 930, 932. The processor 948 can receive input signals indicating positions of the robotic tools 930, 932 and/or the robotic arms 910 (e.g., from position encoders mounted on the robotic arm joints). The processor 948 can receive input signals from the viewer 942 to control positions of the robotic tools 930, 932 and/or the robotic arms 910.

The robotic medical system 900 can include flexible instrument 931. The flexible instrument 931 can include a leader 934 and a sheath 936. A third robotic arm 926 can position the sheath 936. A fourth robotic arm 928 can position the leader 934. The leader 934 can be advanceable within the sheath 936. The leader 934 can include an endoscope and/or endoscopic instrumentation. The leader 934 and/or sheath 936 can be movable by one or more pull wires as described above in relation to the instrument driver 75. The pull wires and/or the third and fourth arm 926, 928 can be operable by the controller 940.

The robotic medical system 900 can enable coordinated movements between one or more of the robotic tools 930, 932 and the leader 934 during a concomitant procedure. During a concomitant procedure, the flexible instrument 931 can be inserted into a natural orifice of a patient 905 (e.g., the nose, mouth, vagina, urethra, rectum, or ear) and one or both of the robotic tools 930, 932 can be introduced into another area of the patient (e.g., percutaneous access into thoracic, abdominal, extra-peritoneal, and/or retro-peritoneal space). For example, during combined percutaneous-endoscopic kidney stone removal, a user can initiate a coordinated mode during which movements of one or more of percutaneous instrument or cameras can maintain a relative position with respect to an endoscope. In another example, during a combined laparoscopic polypectomy, a user can initiate a coordinated mode during which the flexible colonoscopy maintains a relative spacing and/or orientation to the laparoscope and/or laparoscopic tools.

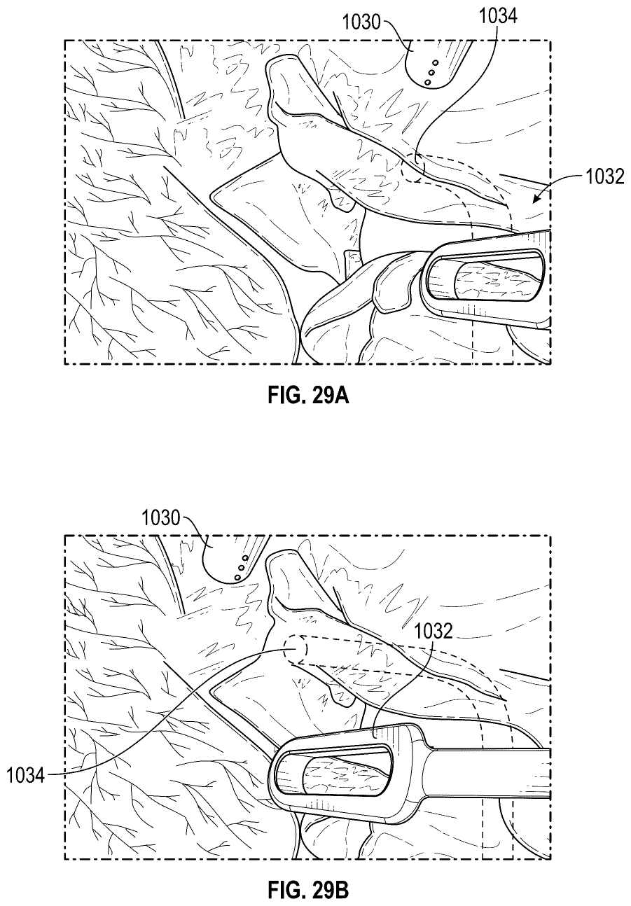

FIG. 29A-B show coordinated movement of a rigid camera 1030, a rigid instrument 1032 and a flexible instrument 1034. The rigid camera 1030 is located within a space within a patient body accessed through an incision. The rigid instrument 1032 is located within the space with the rigid camera 1030. The rigid camera 1030 can provide a view of the rigid instrument 1032. The flexible instrument 1034 can be located in an adjacent space to the space with the rigid camera 1030. The adjacent space can be separated from the space by one or more walls of internal tissue of the patient. For example, the space can be an abdominal space and the adjacent space can be within the colon.

In one implementation, a user can operate to move the rigid camera 1030 in a coordinated motion mode. The rigid instrument 1032 and/or the flexible instrument 1034 can automatically move in coordinated movements to maintain a relative spacing with respect to the rigid camera 1030 (e.g., follower or camera mode). The rigid instrument 1032 can rotate about remote centers of motion (not shown). The flexible instrument 1034 can be advanced/retracted and/or articulated within the adjacent space. Alternatively, the user can move the rigid instrument 1032 and the rigid camera 1030 and the flexible instrument 1034 can automatically move (i.e., advance/retract and/or articulate) in coordinated movements to maintain the relative spacing. In another alternative implementation, the user can move the flexible instrument 1034 and the rigid camera 1030 and/or the rigid instrument 1032 can automatically move in coordinated movements to maintain the relative spacing.

In certain implementations, the coordinated movement of the flexible instrument 1034 or rigid camera 1030 cannot be executed without harm to the patient. For example, the coordinated movement of the flexible instrument may require advancing through a tissue wall to maintain the relative spacing. Accordingly, the robotic medical system can limit the movement of the flexible instrument 1034 and/or the rigid camera 1030. The limited movement can be communicated to a user through haptic feedback, visual alerts, and/or hard stops. In certain implementations, the relative spacing can include a margin of error. As long as the relative spacing stays within the margin of error, the flexible instrument 1034 and/or the rigid camera 1030 can continue to be moved. Once the relative spacing exceeds the margin of error, movement of the flexible instrument 1034 and/or the rigid camera 1030 can be limited.

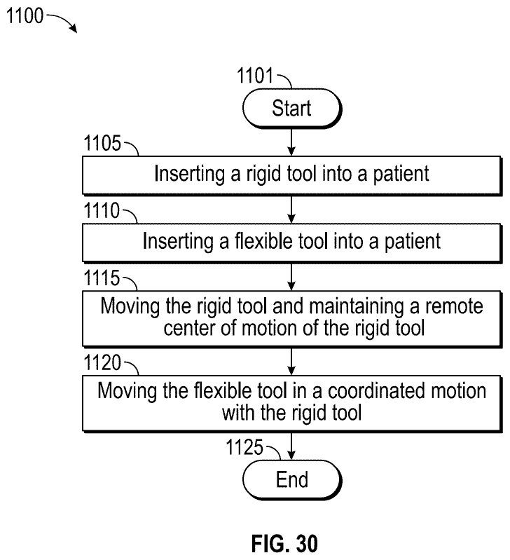

FIG. 30 shows a method 1100 for performing a medical procedure using a robotic medical system. The robotic medical system can include a plurality of robotic arms and a control unit configured to control movement of each of the robotic arms. The method 1100 can start at block 1101. At step 1105 a rigid tool associated with a first robotic arm of the plurality of robotic arms is inserted into a patient through a first port. At step 1110, a flexible tool associated with a second robotic arm of the plurality of robotic arms is inserted into a natural orifice of the patient. At step 1115 a user controls the rigid tool and the first robotic arm using the control unit in a first movement while maintaining a remote center of motion. At step 1120, the flexible tool moves in a coordinated movement with the first movement of the rigid tool. At block 1125 the method 1100 ends.

### 7. Implementing Systems and Terminology.

Implementations disclosed herein provide systems, methods and apparatus for coordinated movement of robotic tools.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions associated with the systems and method for coordinated movement of robotic tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on. ”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

What is claimed is:

1. A robotic surgical system comprising:
a plurality of robotic arms and a control unit configured to control movement of each of the robotic arms;
a first tool associated with a first robotic arm of the plurality of robotic arms; and
a second tool associated with a second robotic arm of the plurality of robotic arms;
wherein a movement of the first robotic arm and the first tool by the control unit results in a coordinated movement of the second robotic arm and the second tool; and
wherein the first robotic arm maintains a first remote center of motion during the movement and the second robotic arm maintains a second remote center of motion during the coordinated movement.

2. The system of claim 1, wherein the first tool is configured to be positioned within a patient through a first port and the second tool is configured to be positioned within the patient through a second port.

3. The system of claim 2, wherein the first remote center of motion is associated with the first port and the second remote center of motion is associated with the second port.

4. The system of claim 1, wherein the second robotic arm and the second tool are not controlled by the control unit during the coordinated movement.

5. The system of claim 1, wherein the first tool comprises a camera.

6. The system of claim 5, wherein the second tool comprises an instrument.

7. The system of claim 1, wherein the movement of the first robotic arm and the first tool by the control unit results in a coordinated movement of a third robotic arm of the plurality of robotic arms and an associated third tool.

8. The system of claim 7, wherein the coordinated movements of the second and third tools maintain a spacing relative to the first tool, and the spacing is at least partially based on reference points on the second and third tools.

9. The system of claim 8, wherein the reference points are formed on tips of the second and third tools.

10. The system of claim 7, wherein the movement of the first robotic arm and the first tool by the control unit results further in coordinated movement of a fourth robotic arm and an associated fourth tool and a fifth robotic arm and an associated fifth tool.

11. The system of claim 1, wherein a center reference point is formed between the second and third tools, such that a spacing between the center reference point and the first tool is maintained during the coordinated movements.

12. The system of claim 1, wherein the control unit is a console comprising a master controller including a first gimbal and a second gimbal and the first gimbal and the second gimbal control the movement of the first robotic arm and the first tool.

13. The system of claim 12, wherein:
the first gimbal controls the movement of the first robotic arm and the first tool and the coordinated movement of the second robotic arm and the second tool, and
the second gimbal controls movement of a third robotic arm and an associated third tool and the third tool maintains a third remote center of motion.

14. A robotic surgery method comprising:
providing a plurality of robotic arms and a control unit configured to control movement of each of the robotic arms;
moving a first tool associated with a first robotic arm of the plurality of robotic arms using the control unit; and
moving a second tool associated with a second robotic arm of the plurality of robotic arms, the movement of the second robotic arm and the second tool coordinated with the movement of the first robotic arm and the first tool; and
maintaining a first remote center of motion during the movement of the first robotic arm and the first tool and maintaining a second remote center of motion during the coordinated movement of the second robotic arm and the second tool.

15. The method of claim 14, further comprising:
positioning the first tool within a patient through a first port; and
positioning the second tool within the patient through a second port.

16. The method of claim 15, wherein the first remote center of motion is at the first port and the second remote center of motion is at the second port.

17. The method of claim 14, further comprising:
moving a third tool associated with a third robotic arm of the plurality of robotic arms, the movement of the third robotic arm and the third tool coordinated with the movement of the first robotic arm and the first tool.

18. The method of claim 17, wherein the movements of the second and third tools maintain a spacing relative to the first tool and the spacing is at least partially based on reference points on the second and third tools.

19. The method of claim 14, further comprising:
controlling the movement of the first tool using a first gimbal and a second gimbal of the control unit.

20. The method of claim 14, further comprising:
controlling the movements of the first robotic arm and the first tool and the second robotic arm and the second tool using a first gimbal of the control unit; and
controlling a movement of a third robotic arm and an associated third tool using a second gimbal of the control unit, the third tool maintaining a third remote center of motion.
